(19) United States
(12) Patent Application Publication
(10) Pub. No.: US 2024/0329044 A1
(43) Pub. Date: Oct. 3, 2024

(54) METHOD FOR PRODUCING A BIOMODIFIED SENSOR FOR DETECTING AND MEASURING PATHOGENS

(71) Applicant: PONTIFICIA UNIVERSIDAD JAVERIANA, Cali (CO)

(72) Inventors: Andrés JARAMILLO BOTERO, Cali (CO); Drochss VALENCIA, Jamundí (CO); Mauricio QUIMBAYA, Cali (CO); Viviana ORTEGA, Cali (CO); Nelson MANCILLA, Cali (CO)

(21) Appl. No.: 18/557,920

(22) PCT Filed: Apr. 27, 2022
(86) PCT No.: PCT/IB2022/053927

§ 371 Date: Oct. 27, 2023

(30) Foreign Application Priority Data
Apr. 27, 2021 (CO) NC2021/0005496

(51) Int. Cl. G01N 33/569; G01N 33/543

(52) U.S. Cl. CPC G01N 33/56983; G01N 33/54353; G01N 33/5438; G01N 2333/165

(57) ABSTRACT

The present invention refers to a method for producing a biomodified sensor for pathogen diagnosis, integrated in an electrochemical cell of three planar interdigitated electrodes (working, reference, and counter) on a flexible substrate for pathogen diagnosis; by modification of the surface of one or more working electrodes with a ligation solution and one or more types of antibodies that selectively bind to one or more types of structural proteins of a pathogen. The invention also refers to electrodes prepared by this method and their applications for pathogen detection. The method of the present invention allows for optimization of the times and conditions known in the art to modify electrodes for detecting biological molecules.

### FIELD OF THE INVENTION

The present invention is in the field of biotechnology, biomedicine and medical diagnostics, in particular in the field of pathogen load detection and measurement devices and their manufacturing, and more particularly it relates to a method for producing a biomodified sensor for pathogen diagnosis, integrated in an electrochemical cell comprising a reference electrode, a counter electrode and one or more planar working electrodes interdigitated on a flexible substrate for selective detection of pathogens at ultra-low concentrations.

### BACKGROUND OF THE INVENTION

The respiratory disease pandemic of COVID-19 caused by severe acute respiratory syndrome coronavirus 2 (SARS-COV-2) is a public health emergency of international relevance, causing a substantial number of cases and deaths worldwide. COVID-19 presents an unprecedented challenge to governments worldwide due to transmissibility and pathogenicity of the virus, the scale of its impact on morbidity and mortality, the uncertainty about the development of long-term immunity in those infected, the current deficiency of treatment options, and the impact on health, economy, and society systems.

Therefore, there is an urgent need for highly selective and sensitive diagnostic solutions for early and rapid identification of infected persons to prevent new infections through individual isolation, breaking the chain of virus spread.

Numerous diagnostic technologies are currently available for detecting SARS-COV-2, among which the diagnostic tool that identifies viral RNA by amplification, reverse transcriptase polymerase chain reaction or RT-PCR, is considered the clinical gold standard. RT-PCR is a sensitive technique but has multiple drawbacks for mass distribution and immediate attention to the pandemic state, such as high costs, long processing times, requirements for equipment and specialized personnel, so it is necessary to develop more efficient methods for detecting COVID-19 positive patients at the point-of-care (POC) or in unstructured environments, including residences, offices, production plants, transportation systems, among many others. In this sense, biosensors represent the best option for rapid and efficient detection. Nowadays, different methods and devices have been developed for detecting viral pathogens.

For example, patent EP3356823B1 discloses a method for preparing a boron-doped diamond (BDD) modified substrate, comprising applying aminobenzoic acid salt on the surface of a BDD substrate which is considered the working electrode, and incubating the binding of biological molecules, particularly specific antibodies to influenza virus Ml proteins, for detection by electrochemical techniques. The document also mentions that the method includes a reference electrode and a counter electrode.

The article “A rapid-response ultrasensitive biosensor for influenza virus detection using antibody modified boron-doped diamond” (Nidzworski, Dawid & Siuzdak, Katarzyna & Niedzialkowski, Pawel & Bogdanowicz, Robert & Sobaszek, Michal & Ryl, Jacek & Weiher, Paulina & Sawczak, Miroslaw & Wnuk, Elżbieta & Goddard, William & Jaramillo-Botero, Andres & Ossowski, Tadeusz. (2017). A rapid-response ultrasensitive biosensor for influenza virus detection using antibody modified boron-doped diamond. Scientific Reports. 7. 10.1038/s41598-017-15806-7) describes a method for obtaining an electrochemical BDD biosensor, surface-functionalized with organic ligands and antibodies against influenza virus M1 protein, and demonstrates rapid virus detection from nasopharyngeal swab samples at concentrations above 5 fg/ml.

In turn, in the article “Highly selective impedimetric determination of Haemophilus influenzae protein D using maze-like boron-doped carbon nanowall electrodes” (Brodowski, Mateusz & Kowalski, Marcin & Skwarecka, Marta & Palka, Katarzyna & Skowicki, Michal & Kula, Anna & Lipinski, Tomasz & Dettlaff, Anna & Ficek, Mateusz & Ryl, Jacek & Dziąbowska, Karolina & Nidzworski, Dawid & Bogdanowicz, Robert. (2021). Highly selective impedimetric determination of Haemophilus influenzae protein D using maze-like boron-doped carbon nanowall electrodes. Talanta. 221. 121623. 10.1016/j.talanta.2020.121623), a method for synthesizing an impedimetric electrochemical biosensor based on maze-like boron-doped carbon nanoparticle (B:CNW) electrode-functionalized with anti-protein D (apD) antibodies for detecting Haemophilus influenzae, is described. The article states that B:CNW electrodes were synthesized in a one-step growth process by microwave plasma-assisted chemical vapor deposition and subsequently modified by electroreduction of diazonium salt on the B:CNW surface, and immobilization of antibodies by zero-length crosslinkers. The document mentions that the developed biosensors showed a highly sensitive and selective response to protein D, with an achieved limit detection of 5.20×10² CFU/ml.

Although methods for producing and modifying biosensors for detecting various pathogens are disclosed in the prior art, there are still challenges in the development and optimization of such technologies, particularly for detecting SARS COV-2, to allow rapid diagnosis, preferably at the POC, and reduce processing costs.

### BRIEF DESCRIPTION OF THE INVENTION

The present invention refers to a method for preparing a biomodified sensor for pathogen diagnosis, integrated in an electrochemical cell comprising a reference electrode, a counter electrode and one or more planar working electrodes interdigitated on a flexible substrate for pathogen diagnosis, comprising modifying the surface of a flexible carbon electrode, depositing on the modified surface a ligation solution and incubating thereon one or more types of antibodies which selectively bind to one or more types of structural proteins of a pathogen.

The carbon electrodes can be screen printed (SPCE, screen printed carbon electrode) using inks of activated carbon, graphene, graphene oxide or a combination thereof, or graphene induced by means of a CO₂ laser (laser induced graphene, LIG).

The present invention refers to biomodified electrodes prepared by any of these methods and their applications for detection and diagnosis of pathogens.

### BRIEF DESCRIPTION OF FIGURES

FIG. 1 Diagram of one embodiment of SPCE working electrode functionalization for SARS-COV-2 protein detection. Bare working electrode, followed by electrodeposition of PABA using cyclic voltammetry; covalent attachment of the carboxyl group from the PABA surface to the SARS-COV-2 antibody for the spike protein(S); passivation of open carboxyl sites on the PABA surface with bovine serum albumin (BSA); and binding of S protein to the antibodies.

FIG. 2 Electrochemical characterization of the SPCE electrode after each functionalization step. A. Bare electrode; B. Electrodeposition of PABA; C. Binding of anti-S protein antibody of SARS-COV-2; D. Passivation of open carboxylic sites with BSA; E. S protein binding to the antibodies.

FIG. 3 Cyclic voltammetry examination of a bare SPCE electrode after the cleaning treatment. The difference between the oxidation and reduction potential peak was approximately equal to 0.11 V and the current intensity of the oxidation and reduction peaks was approximately 10 μA and −15 μA, respectively.

FIG. 4 Cyclic voltammetric electrodeposition of PABA diluted in HCl on the surface of a SPCE. Ten cycles performed at a rate of 100 mV/s.

FIG. 5 Electrochemical characterization of the SPCE electrode functionalized with monoclonal antibodies (SPCE-mAb) at different stages of functionalization. A. Binding of SARS-COV-2 to S protein. B. Negative control using Epstein-Barr virus (EBV) proteins. C. Negative control using H1N1 influenza virus proteins.

FIG. 6 Control impedance spectra at each functionalization step, recorded in K3 Fe(CN)₆/1×PBS, 0.01 M phosphate ion (PO₄⁻³) at pH 7.4, measured at open circuit potential (OCP). OCP vs Ag/AgCl/0.1 M KCl, Edc vs OCP. Electrodes were electrochemically modified with PABA at 2 cycles, and subsequently BSA was immobilized in 0.5M MES buffer pH 6.0.

### Electrodes: A. C5H6; B. C6H6; C. C9H6.

FIG. 7 Control impedance spectra at each functionalization step, recorded in K3 Fe(CN)₆/1×PBS, 0.01 M phosphate ion (PO₄⁻³) at pH 7.4, measured at open-circuit potential (OCP). OCP vs Ag/AgCl/0.1 M KCl, Edc vs OCP. The electrodes were electrochemically modified with PABA at 2 cycles, modified with monoclonal antibodies and subsequently blocked with BSA in 0.5M MES buffer pH 6.0. Electrodes: A. F14H3; B. F15H3; C. F16H3.

FIG. 8 Control impedance spectra at each functionalization step, recorded in K3 Fe(CN)₆/1×PBS, 0.01 M phosphate ion (PO₄⁻³) at pH 7.4, measured at open-circuit potential (OCP). OCP vs Ag/AgCl/0.1 M KCl, Edc vs OCP. Electrodes were electrochemically modified with PABA at 2 cycles, modified with monoclonal antibodies, and subsequently buffered with BSA in 1×PBS buffer (0.01M phosphate ion and pH 7.4). Electrodes: A. B19E5; B. B20E5; C. B21E5.

FIG. 9 Linear calibration curve of a biomodified electrode array as a function of the change in charge transfer resistance Rct (absolute and relative in percent) for different concentrations of SARS-COV-2 spike protein.

### DETAILED DESCRIPTION OF THE INVENTION

For purposes of interpreting terms used throughout this document, their usual meaning in the technical field shall be considered, unless a particular definition is included, or the context clearly indicates otherwise. In addition, terms used in the singular form shall also include the plural form.

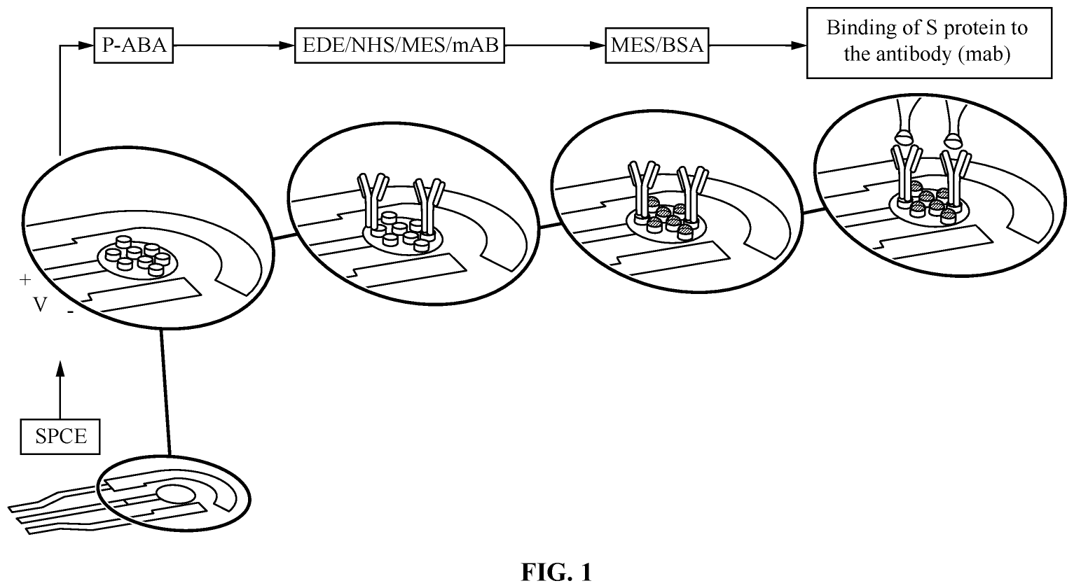

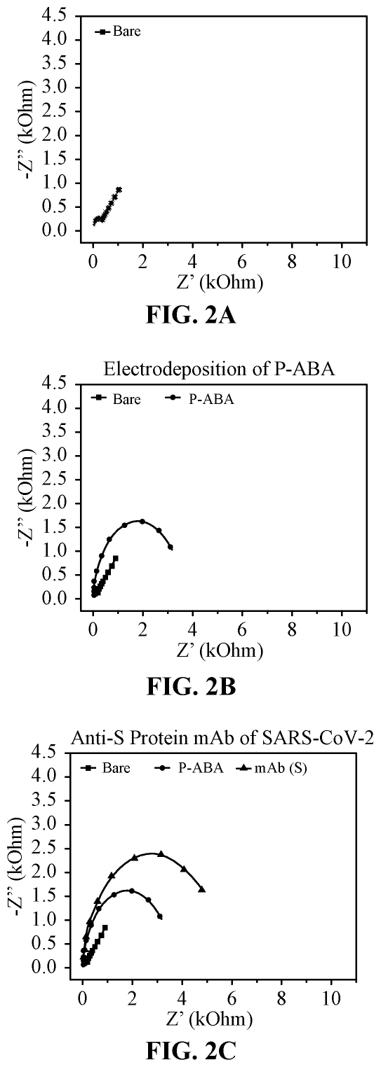

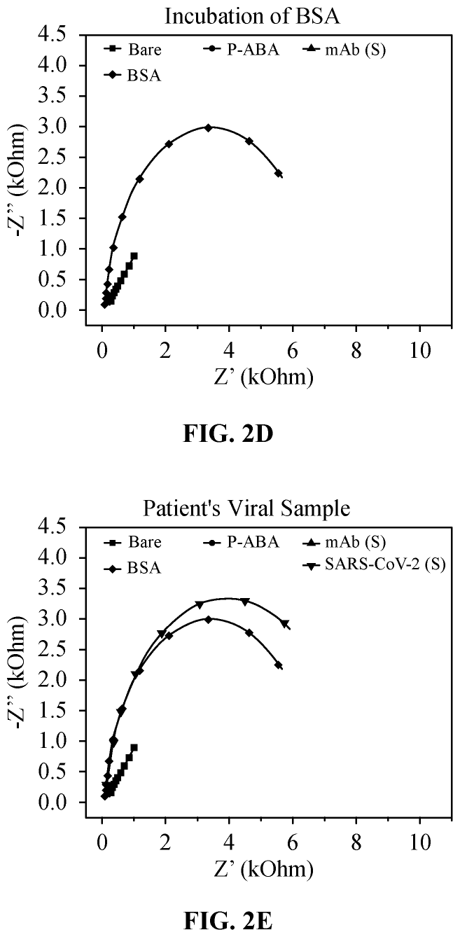

The present invention refers to a method for preparing a biomodified sensor for pathogen diagnostics, integrated in an electrochemical cell that comprises a reference electrode, a counter electrode and one or more planar working electrodes interdigitated on a flexible substrate for pathogen diagnostics, that comprises the steps of: (i) modifying the surface of an electrode with a solution of an organic acid or a salt thereof; ii) depositing on the modified surface a ligation solution for activation of the carboxylic group in the organic acid layer or salt thereof comprising 1-ethyl-3-(3-dimethylaminopropyl) carbodiimide (EDC), a 2-morpholino ethane sulfonic acid buffer (MES) and N-hydroxysuccinimide (NHS) or N-hydroxysulfosuccinimide (sulfo-NHS); and iii) depositing on the surface containing the activated ligation solution, a solution comprising one or more types of antibodies that selectively bind to one or more types of structural proteins of a pathogen; wherein the electrode is selected from an electrode printed on a flexible polymer film by screen printing with an ink of carbon allotrope materials or a graphene electrode induced by CO₂ laser from a polyimide film or Kapton tape. A schematic illustration of one embodiment of the method is shown in FIG. 1.

For the purposes of the present invention, screen printed carbon electrodes (SPCE) on a polymer film (e.g., polyethylene-PET), or laser-induced graphene electrodes (CO₂) of a polyimide film, (LIG—Laser-Induced Graphene Electrodes) can be commercially available electrodes or those prepared by methods known in the art.

For purposes of the present invention, the term “carbon allotrope material” includes, but is not limited to diamond, graphite, graphene, amorphous carbon, carbon nanotubes, carbon nanobuds, glassy carbon and mixtures thereof.

Screen-printed electrodes with allotropic carbon material can be prepared by the thick film printing technique as described, e.g., in the articles by Wan et al, Ye et al or Soares et al (Laser induced graphene for biosensors. Sustainable Materials and Technologies. 25. e00205. 10.1016/j.susmat.2020.e00205: Laser-Induced Graphene. American Chemical Society 2018, 51, 7, 1609-1620. https://doi.org/10.1021/acs.accounts.8b00084; Laser-Induced Graphene Electrochemical Immunosensors for Rapid and Label-Free Monitoring of Salmonella enterica in Chicken Broth. 2020. Sensors. 10.1021/acssensors.9b02345).

Graphene electrodes can be obtained by laser reduction (CO₂) of a polyimide polymer film according to methods described, e.g., in the articles by Han et al, and Dominguez-Renedo et al (Multifunctional Flexible Sensor Based on Laser-Induced Graphene. 2019. Sensors. 19. 3477. 10.3390/s19163477; Recent developments in the field of screen-printed electrodes and their related applications. September 2007 Talanta 73 (2): 202-219, DOI: 10.1016/j.talanta.2007.03.050).

### Electrode Cleaning Treatment and Electrochemical Electrode Characterization

Before modification and functionalization, the electrodes must be subjected to a cleaning process to remove or reduce residues of the polymeric component used as a binding agent in carbon inks for screen printing, since this interferes with the electrochemical measurement, or to remove contaminants resulting from contact with environmental conditions, such as humidity, oxygen species, nitrogen, carbon compounds, etc.

The cleaning process can be performed by methods known in the art, such as thermal methods, mechanical polishing, solvents, or alkaline solutions. It must be ensured that the chemical agent used does not degrade the polymer substrate, e.g., PET of SPCEs, or polyimide of LIGs.

Regarding the cleaning by solvents or alkaline solutions with or without activation potentials, cleaning agents include, but are not limited to alcohols, such as analytical-grade methanol (CH₃OH); bases such as sodium hydroxide (NaOH), and other compounds such as sulfuric acid (H₂SO₄), potassium chloride (KCl), sodium bicarbonate (NaHCO₃), and sodium carbonate (Na₂CO₃).

In an embodiment, the cleaning of the SPCEs is performed by rubbing in a circular manner the working electrode with a swab impregnated in analytical-grade methanol, performing a moderate, constant, and uniform pressure, preventing the methanol solution from coming in contact with the Ag/AgCl reference electrode to avoid its deterioration. This procedure is performed twice using new swabs.

In an embodiment, cleaning of the SPCEs is performed by immersing the electrodes in a 3M NaOH solution for 1 h, thereby increasing the C/O ratio at the electrode surface.

Subsequently, the behavior of the electrodes towards the redox active species is characterized. The characterization can be performed by techniques known in the technical field, such as cyclic voltammetry, electrochemical impedance spectroscopy (EIS) or combinations thereof. The electrode can be re-characterized after each modification step, or only in the last step to obtain a blank reference measurement (i.e., without the analyte).

In a particular embodiment, the cleaning quality is verified by cyclic voltammetry, which must account for a reduction in the charge transfer resistance (Rct) of the working electrode.

Modification of an Electrode Surface with an Organic Acid or a Salt Thereof

Treatment of the electrode surface with an organic acid or a salt thereof is performed to generate a base layer for anchoring biological molecules, such as antibodies, bovine serum albumin, and other proteins. As used herein, the organic acids used include, but are not limited to para-aminobenzoic acid (PABA), pyrene butyric acid (PBA) and 1-pyrenobutanoic acid succinimidyl ester (PBSE), and salts thereof.

In a particular embodiment, activated carbon, amorphous carbon, glassy carbon, and diamond electrodes, and all those with SP3 hybridization state, are modified with ligands containing an amino group at one end to couple by deamination the aromatic ring carbon 4 to the carbon surface and an activated carboxylic group at the other end to bind proteins (antibodies). An example of such ligands is PABA.

In another particular embodiment, graphene electrodes, and all those with SP2 hybridization state, are modified with ligands having at one end any type of aromatic ring that allows the linking by pi-orbital stacking (‘pi-stacking’) with the graphene lattice, and at the other end a carboxylic group to bind proteins by peptide bonds, particularly with the lysine residues thereof. Examples of such ligands are PBA and PBSE.

In a particular embodiment of the invention, the modification is performed using diazonium salts of PABA. The synthesis of these salts is performed by treatment of primary aromatic amines with sodium nitrite in the presence of an inorganic acid, at a temperature between −2°° C. to 5° C., which is known as diazotization.

It is necessary that the reaction be performed with a primary amine and that the amine be aromatic because the compound to be obtained is very unstable. Therefore, the aromatic ring will confer a certain degree of stability. In a particular embodiment, the reagent used for this process is sodium nitrite in excess of a strong mineral acid such as hydrochloric acid at low temperature, thus, an ice bath is used so that the temperature is maintained between 0° C. and 5° C. during the synthesis.

Surface modification of the electrode with diazonium salts of PABA, can be performed by electrodeposition, which involves the electroreduction of amine groups on the diazonium salts to form covalent bonds with the electrode carbon surface. This process is performed by the cyclic voltammetry technique.

PABA is a natural compound of low molecular weight that can be deposited with a high-density surface coating, in a monolayer or in multiple layers, on a carbon surface in the SP3-hybridized state. Electrodeposition deamination of PABA using low potentials (1-2 V) creates stable covalent linkers between the SP3-hybridized carbons on the electrode surface. Activation by the open end of the carboxyl group of PABA also allows the binding of antibodies or proteins by means of peptide bonds, typically to lysines.

In a particular embodiment of the invention, the modification is performed using PBA or PBSE, by non-covalent tethering to the graphene lattice, thereby obtaining a self-assembled monolayer that avoids electroreduction of the ligand on the surface. The pyrene group binds to the graphene surface through stable non-covalent π-π-type interactions and presents a carboxyl (or NHS-ester ligand in the case of PBSE) for antibody coupling at the free end for biofunctionalization.

The 1-pyrene butyric acid (PBA) is used as the heterobifunctional ligand to anchor the antibodies on graphene. The use of PBA does not disrupt the conjugation of graphene sheets and improves their stability. PBA consists of a pyrene group containing π electrons and a carboxylic group that is used to functionalize the graphene surface through π-π-type orbital stacking and hydrophobic interactions. PBA pyrene units interact strongly with the graphene surface without altering the electronic transmissivity of the material. In an embodiment, an EDC/NHS/MES solution is used to activate the SPCE surface and form peptide binding between the carboxylic groups on the PBA and the —NH₂ groups of the specific antibodies incubated for one hour. In another embodiment, antibodies are incubated for 24 hours after activation with EDC/NHS.

### Deposition of the Ligation Solution on the Modified Surface

The ligation solution deposited on the modified electrode surface comprises 1-ethyl-3-(3-dimethylaminopropyl) carbodiimide (EDC), a 2-morpholino ethane sulfonic acid (MES) buffer and N-hydroxysuccinimide (NHS) or N-hydroxysulfosuccinimide (sulfo-NHS).

In an embodiment, the ligation solution comprises EDC and NHS, dissolved in the sodium salt buffer solution of 2-morpholino ethane sulfonic acid, (MES Na).

In another embodiment, the ligation solution comprises EDC and sulfo-NHS, dissolved in MES Na.

EDC reacts with the carboxyl group at the free end of the ligand used (electrodeposited monolayer on the electrode surface) to form an active O-acylisourea intermediate. EDC is readily displaced by nucleophilic attack of the antibody primary amine groups (—NH₂) which are mainly on the lysine residues in the heavy chains (or at the antibody N-termini) of the antibody FC region. They form an amide linker with the original carboxyl group and release an EDC by-product in the form of a soluble urea derivative. The O-acylisourea intermediate is unstable in aqueous solutions, therefore, if it does not find and react with an amine, hydrolysis of the intermediate occurs resulting in regeneration of the carboxyl groups and release of a urea with no N-substituent.

In another particular embodiment, sulfo-NHS is added as it stabilizes the reactive intermediate to the amine converting it into an amine reactive sulfo-NHS ester, thereby increasing the efficiency of EDC-mediated coupling reactions.

NHS, or its water-soluble analog sulfo-NHS, is included in the EDC coupling protocol to improve efficiency or create more stable intermediates than O-acylisourea (makes it an amine-reactive sulfo-NHS ester). In this case, the O-acylisourea complex that has not been hydrolyzed or conjugated into a peptide bond is coupled to the NHS by the carboxylic groups to form an NHS ester that is considerably more stable than the O-acylisourea intermediate, while achieving efficient conjugation of the primary amines to the lysine residues of the antibody, at physiological pH.

Sulfo-NHS esters are hydrophilic active groups that readily couple with amines on target molecules with the same specificity and reactivity as NHS esters. Unlike NHS esters that are relatively insoluble in water and must first be dissolved in an organic solvent before being added to aqueous solutions, sulfo-NHS esters are relatively soluble in water and have a longer lifetime and hydrolyze more slowly in water. However, activation with NHS decreases the water solubility of the modified carboxylate molecule, whereas activation with sulfo-NHS preserves or increases the water solubility of the modified molecule pursuant to the charged sulfonate group. Although NHS or sulfo-NHS esters are sufficiently stable, prepared in two-step reaction processes, both groups will hydrolyze within hours or minutes, depending on the water content and pH of the reaction solution.

Therefore, in order to maximize amine modification and minimize hydrolysis effects, a high concentration of protein or other target molecule must be maintained in the reaction medium.

Water-insoluble linkers containing NHS esters can be reacted in organic solvents, overcoming the problem of hydrolysis, provided the target molecule is soluble and stable in such environments. For non-aqueous reactions, an organic base (proton acceptor), such as triethylamine or 4-(dimethylamine) pyridine (DMAP), is added.

EDC coupling is most efficient under acidic conditions (pH 4.5) and shall be performed in buffers that do not have carboxyl groups or amines other than those of interest, such as MES buffer. Phosphate buffers (e.g., PBS) and neutral pH conditions (up to 7.2), are compatible with the chemistry of this reaction, however they have lower efficiency, thus, it is necessary to increase the amount of EDC in the reaction solution to compensate for the lower efficiency or increase the incubation time.

The activation reaction with EDC and sulfo-NHS is most efficient at pH 4.5 to 7.2. For best results in two-step reactions, the first reaction is performed in MES buffer (or other buffer without amine or carboxylate) at pH 5 to 6, and then pH is increased to 7.2 to 7.5 with phosphate buffer (or other buffer than amine buffer) immediately prior to the reaction to the amine-containing molecule.

In an embodiment of the invention, EDC and NHS or sulfo-NHS are in solution in a 1:1 ratio.

In a particular embodiment, between 3 μl to 5 μl EDC and NHS or sulfo-NHS solution are deposited on the electrode.

In a particular embodiment of the invention, the ligation solution is incubated on the electrode surface for between 1 to 2 hours, at a constant temperature between 4° C. and 25° C.

Electrode Modification with One or More Antibodies

After incubation with the ligation solution, the electrodes are modified by immobilization of one or more types of antibodies which would be selective to one or more types of antigens. The antibodies of the invention are selected from human, recombinant, humanized and/or chimeric monoclonal antibodies;human, recombinant, humanized and/or chimeric polyclonal antibodies; aptamers or mixtures thereof.

In an embodiment, at least one antibody aimed at one or more surface antigens of the pathogen, and/or at least one antibody aimed at one or more genetically conserved antigens, is employed.

In a particular embodiment, the antibodies selectively bind to one or more structural proteins of the one or more target pathogens.

In an embodiment, antibodies are primarily selected from antibodies that bind external proteins that mediate pathogen entry into host cells, followed by antibodies that bind genetically conserved proteins among families, variants, species, genera, or strains, as appropriate. In a particular embodiment, a mixture of antibodies that bind to external proteins, as well as antibodies that bind to genetically conserved proteins, are employed to improve selectivity in detection of the specific pathogen.

In an embodiment, the pathogen is a virus, more specifically a human virus, more specifically a human respiratory virus. Examples of human respiratory viruses include, but are not limited to, respiratory syncytial virus, parainfluenza viruses, methaneumoviruses, rhinoviruses, coronaviruses (SARS-COV viruses, SARS-COV-2 viruses, MERS-COV viruses), adenoviruses, and bocaviruses.

In a particular embodiment, the virus is selected from among the coronaviruses (SARS-COV virus, SARS-COV-2 virus, MERS-COV virus). In a more particular embodiment, the virus is SARS-COV-2 virus.

In an embodiment, the antibody selectively binds to one or more proteins selected from the group comprising spike protein(S), nucleocapsid protein (N), membrane protein (M) or envelope protein (E) of SARS-COV-2 virus or mixtures thereof.

In an embodiment, a mixture of antibodies directed to proteins from different genetic variants of a pathogen, or from different strains of a pathogen, or from different species of a genus, or from different genera of pathogens, is employed. In a particular embodiment, equal ratios of each antibody directed against a particular protein of each specific strain or variant or in ratios equivalent to the prevalence of one strain versus another in a region, are employed. For example, given antigen complementarity (conserved primary sequence regions between strains), in one case the nucleocapsid mutates less and may anchor proteins from the mutant that are not likely to be readily detected from that mutant's spike. In these cases, sensitivity may be compromised, but the limit of detection is increased, and the instrument remains selective.

In a particular embodiment, a biomodified working electrode is included for each genetic variant (with antibodies specific thereto), without altering the reference and counter electrode. In this embodiment, the instrument is a multiplexed system between working electrodes, using the same reference and counter electrodes to avoid sacrificing selectivity and sensitivity that may be affected when a single electrode is modified with several different antibodies.

For purposes of the present invention, the one or more antibodies are deposited on the surface of the working electrode at a concentration of 8 to 15 μg/ml, dissolved in a buffer solution at a concentration of 1×, at pH between 4 to 6. The buffer solution employed may be any known in the art. In a particular embodiment, the buffer solution is phosphate buffered saline (PBS).

In a particular embodiment, the solution containing the one or more antibodies is incubated on the surface of the working electrode for 1 to 2 hours, at a constant temperature between 4° C. and 25° C.

Subsequently, the surfaces not bound by the antibody on the electrode shall be washed, dried, and coated. Washing is performed with distilled water, low concentration PBS solution, distilled-deionized water, or saline solution, among others. Also, drying is performed at room temperature or using a low-flow hot air stream.

As used herein, unless otherwise stated, ambient temperature refers to temperatures in the range of 20 to 25° C.

Coating of the surfaces not bound by the antibody with blocking agent is performed by incubating the electrode with solutions known in the technical field for this purpose, which include but are not limited to bovine serum albumin (BSA) solution, at a concentration between 1% to 5%, for 1 to 2 hours at a constant temperature between 4° C. and 25° C.

### Electrode

In a further aspect, the present invention refers to a biomodified electrode according to the method described in the present invention, comprising a screen-printed electrode with allotropic carbon material in SP₃ hybridization state on a polymer film, or a surface-modified graphene electrode with a monolayer of an organic acid or a salt thereof and bound to one or more types of antibodies that selectively bind to one or more types of proteins of a pathogen.

### Uses

In yet another aspect, the present invention refers to the use of the biomodified electrode obtained by the method described herein, in applications for detecting pathogens deposited on surfaces, contained in food, present in nasopharyngeal fluids, respiratory tract fluids, intestinal tract fluids, or saliva for clinical diagnosis and monitoring.

The present invention is set forth in detail through the following examples, which are provided for illustrative purposes only and not with the purpose of limiting its scope.

### EXAMPLES

### Example 1. Method for Functionalizing Screen-Printed Carbon Electrodes (SPCEs)

As used herein, the electrodes are printed or produced in a matrix arrangement on the polymer sheet. Each electrode is identified by a four-digit alphanumeric code, which indicates its position on the polymer sheet, wherein the first digit is a capital letter corresponding to the column, the second digit is a number corresponding to the row, the third and fourth digits are the letter H and a sequence integer, which indicate the polymer sheet. For example, C9H6 corresponds to the code of the electrode located in column 3, row 9 of the printed sheet number 6.

### Electrode Pretreatment

The surface of the working electrode was cleaned with a swab soaked in analytical-grade methanol, rubbing the entire electrode in a circular manner, exerting constant pressure. The process was performed twice.

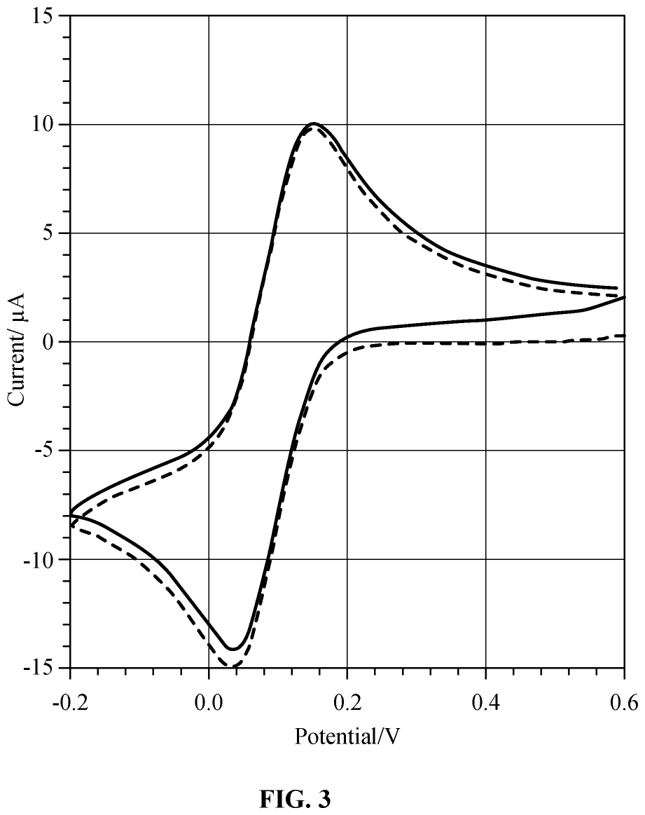

Cyclic voltammetry examinations were performed on the bare SPCE electrode after the cleaning treatment. The difference between the oxidation and reduction potential peak was approximately equal to 0.11 V and the current intensity of the oxidation and reduction peaks was approximately 10 μA and −15 μA, respectively (FIG. 3).

In order to follow the changes in electrode resistance at each step of the electrode modification, electrochemical impedance spectra (EIS), measured at open circuit potential (OCP) of the redox reaction: [Fe(CN)₆]³⁻/[Fe(CN)₆]⁴⁻, (Fe³⁺/Fe²⁺), were recorded. For the bare SPCE electrode, after the cleaning treatment, the actual impeda value was approximately 5000Ω.

Electrochemical measurements were performed by CV and EIS after each modification step. The parameters of each technique are shown in Table 1.

TABLE 1
Electrochemical measurement parameters

| Cyclic Voltammetry Parameters | | | Impedance Spectroscopy Parameters | | |
|---|---|---|---|---|---|
| Equilibrium t | 3 | s | Equilibrium t | 40 | s |
| Home E | 0.6 | V | Scan Type | Fixed | |
| Vertex E1 | −0.2 | V | E dc | 0.0 | V |
| Vertex E2 | 0.6 | V | E ac | 0.01 | V |

| | | | | |
|---|---|---|---|---|
| Step E | 0.01 | V | Frequency Type | Scanning |
| Scan Rate | 0.05 | V/s | No. of Frequencies | 53 = 8.7/dec |

| | | | | |
|---|---|---|---|---|
| Number of Scans | 2 | Max. Frequency | 20,000.0 | Hz |

| | | |
|---|---|---|
| Min. Frequency | 0.02 | Hz |

### Synthesis of the PABA Diazonium Salt

20 mg of PABA were dissolved in 2 ml 0.1M HCl, the solution was stirred for 15 min using ultrasound and allowed to cool down at 0° C. Next, 2 ml more 0.1M HCl was added to the mixture and the mixture was stirred for 15 min using ultrasound until complete dissolution. Finally, a solution of 25 mg NaNO₂ dissolved in 3 ml of 0.1M HCl was added dropwise over 30 minutes. It was stirred at 0° C. for 10 minutes to obtain the diazonium salt. The temperature was maintained between 2° C. and 5° C. using ice bath throughout the synthesis process.

Modification of the SPCE Electrode Surface by Electrografting with PABA Diazonium Salt

Initially, the three electrodes (working, auxiliary and reference) were covered with 50 μl of diazonium salt. The electrodeposition of the diazonium salt was performed by cyclic voltammetry. Two potential sweeps were performed between 0.2 and −0.6 V, at a sweep rate of 0.1 V/s.

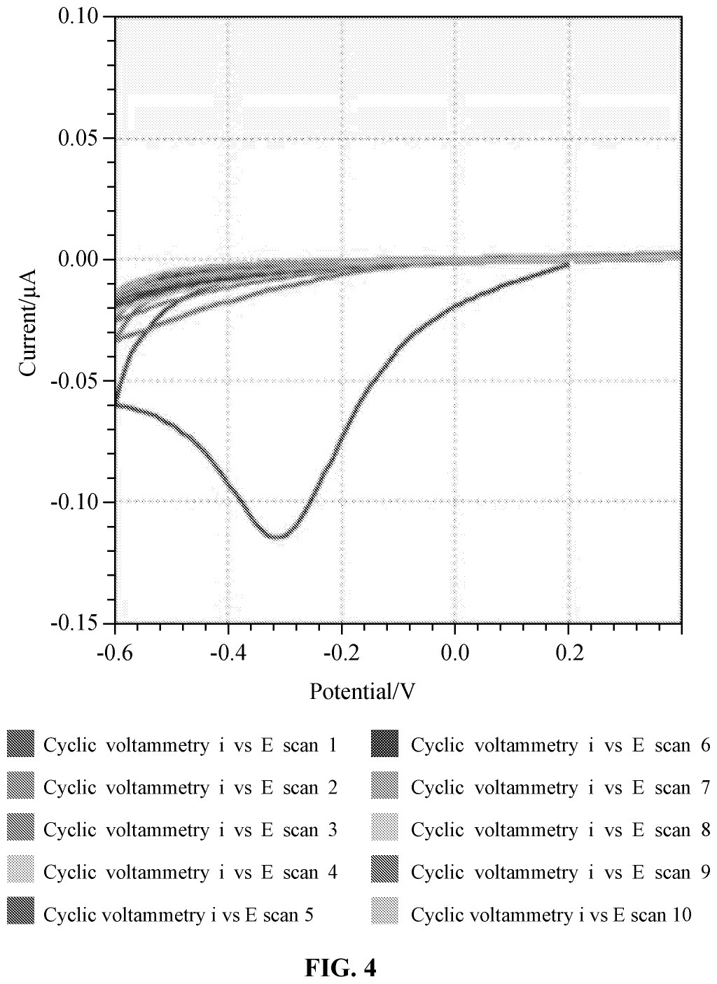

The modified electrodes were washed with type-1 water and dried under a stream of cold air. Cyclic voltammograms of reduction and deposition of the PABA diazonium salt in a dilute HCl solution were performed on the surface of the SPCE electrode (FIG. 4). In the first cycle, an irreversible reduction peak was observed at −0.3V measured at open circuit potential (OCP), OCP vs Ag/AgCl, corresponding to the electroreduction band of the diazonium salt and the production of an aryl radical that in the next step forms a covalent bond on the SPCE electrode surface. In the subsequent scans (from 2 to 10), a decreasing current was observed, and in the last scans, the reduction peaks are not present. The above is proof of surface saturation and suggests that a thin layer of acid functional groups was obtained.

Diazonium Salt Modification with Ligation Solution and Antibody Immobilization

An EDC solution was prepared by dissolving 19.17 mg of EDC in 1 ml of 0.5 M MES Na buffer at pH 6.0. In turn, a solution of NHS was prepared by dissolving 11.51 mg of NHS reagent in 1 ml of 0.5 M MES Na buffer at pH 6.0.

A mixture of the EDC/NHS solutions was prepared in a 1:1 ratio and 4 μl of this solution were deposited on the surface of the working electrode. It was incubated for one hour at 5° C.±0.5° C. Subsequently, the electrode was washed with approximately 200 μl of 0.5 M MES buffer pH 6.0 and dried at 20° C.±1° C.

Then, an anti-spike or anti-nucleocapsid antibody solution was prepared at 10 μg/ml dissolved in 1×PBS, 0.01 M phosphate ion (PO₄⁻³), pH 7.4, and 4 μl of this solution were deposited on the active surface of the SPCE working electrode. It was allowed to react for one hour at 4° C. in water-saturated atmosphere. The electrode was then washed with approximately 5 ml 1×PBS solution, and dried at 20° C.±1° C. The antibodies used are listed in Table 2.

TABLE 2
Anti-SARS-CoV-2 antibodies tested

| Supplier | Code | Blank | Isotype/Origin |
|---|---|---|---|
| SinoBiological | 40591-MM43 | Spike protein S1 | Mouse |
| | | (SARS-CoV-2) | monoclonal IgG1 |
| SinoBiological | 40592-R001 | RBD spike protein | Rabbit |
| | | (SARS-CoV-2) | monoclonal IgG |
| SinoBiological | 40143-R004 | Nucleocapsid | Rabbit |
| | | protein (SARS- | monoclonal IgG |
| | | CoV-2) | |
| Abcam | ab272420 | Spike protein | Mouse |
| | | (SARS-CoV-2) | monoclonal |
| | | | IgG2b |
| Abcam | ab272504 | Spike protein | Rabbit polyclonal |
| | | (SARS-CoV-2) | IgG |
| Abcam | ab271180 | Nucleocapsid | Rabbit |
| | | protein (SARS- | monoclonal IgG |
| | | CoV-2) | |

The functionalized SPCE-mAb electrode was characterized by CV and EIS recorded in 50 μl of 1 mM K₃Fe (CN)₆ solution. Supporting electrolyte: 1×PBS, 0.01 M phosphate ion (PO₄⁻³), measured at open circuit potential (OCP). OCP vs Ag/AgCl, E_{dc} vs OCP. EIS electrochemical impedance spectroscopy measurements, were performed in a frequency range from 20 kHz to 0.02 Hz, covering 53 points and with a amplitude of 10 mV of the AC signal.

Blocking Active Sites Not Bound by Antibody with BSA (Passivation)

A 1% BSA solution in 0.5 M MES buffer pH 6.0 was prepared at 1%. On the SPCE working electrode, 4 μl of the BSA solution were deposited. The reaction was maintained for 1 h at 5.0° C.±0.5° C. Subsequently, the surface was washed with 5 ml of 1×PBS and dried at 20° C.±1° C.

The functionalized SPCE-mAb electrode was characterized by CV and EIS recorded in 50 μl of 1 mM K₃Fe (CN)₆ solution. Supporting electrolyte: 1×PBS, 0.01 M phosphate ion (PO₄⁻³), measured at open circuit potential (OCP). OCP vs Ag/AgCl, Ede VS OCP. EIS electrochemical impedance spectroscopy measurements were performed in a frequency range from 20 kHz to 1 Hz, covering 53 points and with a 10 mV AC signal amplitude.

Finally, the prepared electrode surface was washed with plenty of type-1 water (approximately 5 ml) and stored at 5° C.±0.5° C.

### Determination of Protein Binding to the Modified Electrode: SPCE-PABA-EDC-NHS (MES)-AntiCoV2-Sp-BSA (MES)

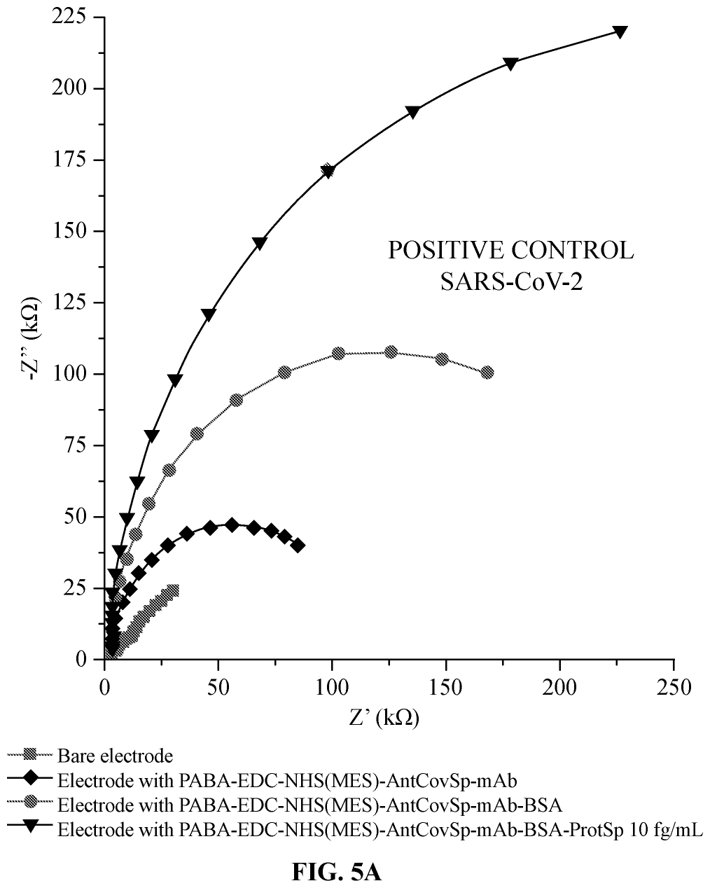

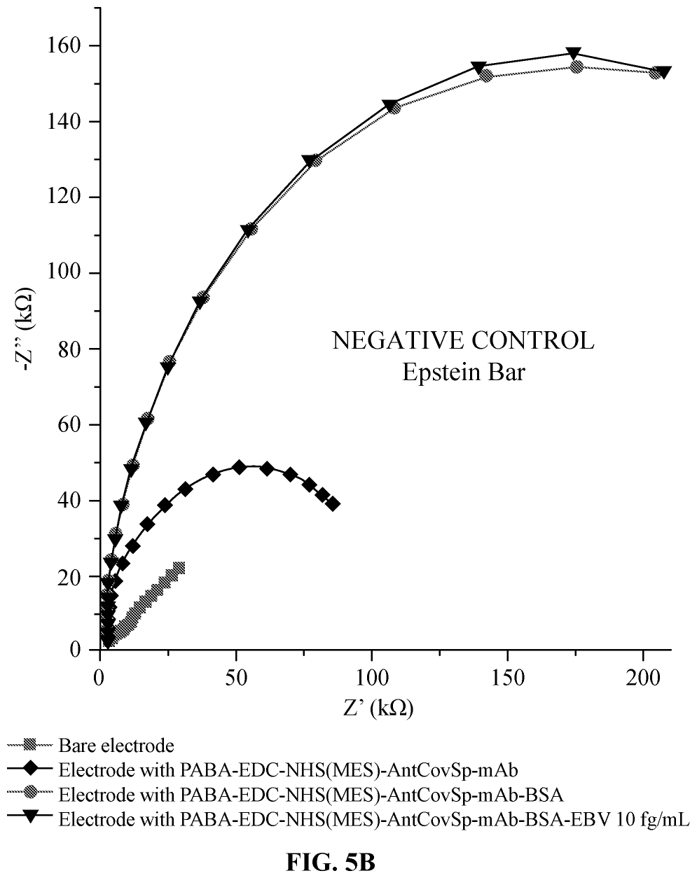

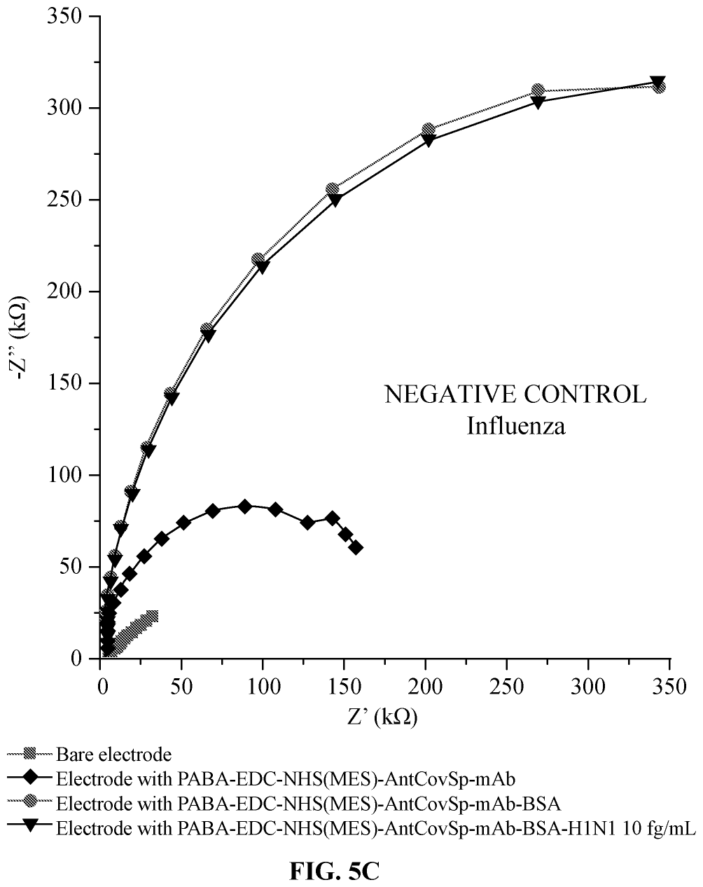

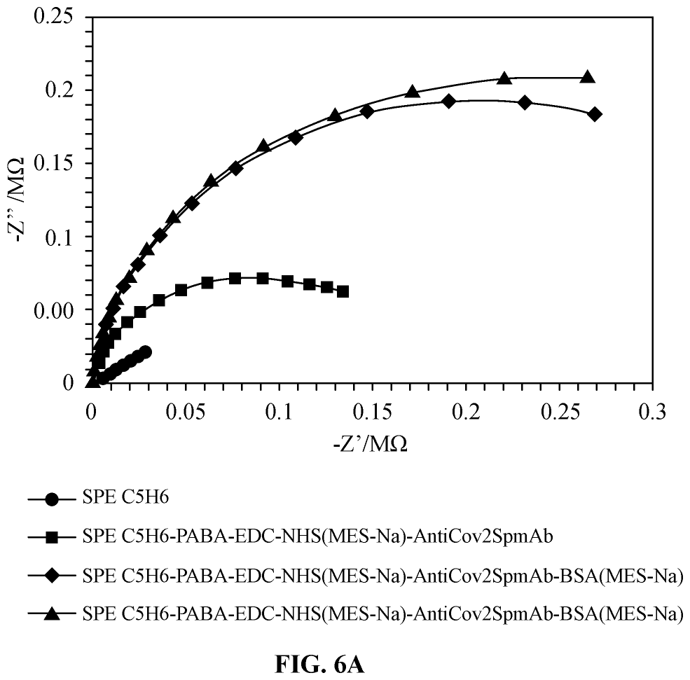

Protein linker binding to the modified electrode was determined using recombinant proteins and patient samples. For validation using recombinant proteins, recombinant spike (and nucleocapsid) protein dilutions were prepared at different concentrations (1, 5, 10, 20, 20, 50, 75 and 100 fg/ml in 1×PBS). Then, 3 μl of each dilution were added onto the modified working electrode of different SPCEs and each was incubated at 4° C. for 10 min. Each electrode was then washed with 1× PBS buffer and characterized by EIS to demonstrate the incremental change in Rct as a function of concentration change. FIGS. 5A to 5C show the binding of the electrodes functionalized with Anti-S protein Mab to recombinant proteins at a concentration of 10 fg/ml, but not to proteins from other respiratory viruses such as Epstein-Barr virus (EBV) and H1N1 influenza virus.

For validation with patient samples, nasopharyngeal fluid matrix (or saliva) was mixed in Triton-X100 at 0.25-0.5%, and 3 μl of the solution were incubated on the modified working electrode at 4° C.±0.5° C. for 10 minutes. Subsequently, each electrode was washed with 1×PBS buffer and characterized by EIS. The difference in Rct with respect to the blank measurement determined the patient's viral load. Positivity diagnosis was established with an increase of more than 10%.

Electrochemical measurements of cyclic voltammetry (CV) and electrochemical impedance spectroscopy (EIS) were performed using a PalmSens 4 galvanostat potentiostat coupled to PStrace software, with an adapter for disposable flexible electrodes. For all electrochemical measurements, a solution of K₃Fe(CN)₆ prepared in 0.01M PBS phosphate-buffered saline (1×PBS) previously degassed was used.

### Example 2. Laser-Induced Graphene Electrode Functionalization (LIG) Method

For the modification of LIG electrodes, the same steps of Example 1 were used, except for the modification of the electrode surface, which was performed with pyrene butyric acid, according to the following procedure:

On the graphene surface, 10 to 20 μL of 5 nM butyric pyrene acid were incubated for 2 hours under a humid atmosphere at room temperature. After rinsing with N,N-Dimethylformamide (DMF) and isopropyl alcohol (IPA) and deionized water, and drying under air flow, the electrodes were incubated with 10 ml a mixing solution containing 0.4 M EDC and 0.1 M sulfo-NHS in 0.025 M MES (pH 6.5) for 35 min at room temperature under humid ambient conditions. The electrode surfaces were drip-coated with a solution of specific antibodies against SARS-COV-2 spike and/or nucleocapsid proteins at a concentration of 10 μg/ml and incubated for 3 h at room temperature. Non-linker surfaces were then inactivated by the antibodies with 1.0% BSA prepared in 0.01 M PBS. The combination of antibodies on the same working electrode, particularly anti-spike and anti-nucleocapsid allow for coverage of genetic variants in the virus spike protein, as the region encoding the nucleocapsid protein is more conserved between variants.

### Example 3. Determination of the Apparent Electrode Surface Coverage Parameter θ (%) for Electrodes Functionalized with BSA (MES 0.5 M pH 6.0) vs BSA (1×PBS)

In order to conduct the contrast examination of the functionalization of SPCE electrodes employing MES, (BSA (0.5M MES pH 6.0) vs BSA (1×PBS, 0.01 M phosphate ion (PO4-3) at pH 7.4)), the “apparent electrode surface coverage θ (%)” parameter, estimated from measurements and equivalent circuit examination of Electrochemical Impedance Spectroscopy (EIS) data, was used on SPCE electrodes functionalized to BSA.

Data under consideration correspond to EIS measurements performed immediately after BSA incubation (first day) and EIS measurements as BSA blank or reference performed on the following day (second day).

The collected electrochemical impedance spectra (EIS) were analyzed using the equivalent electrical circuit R₁[Q₁(R_{ct}W₁)], which contains the electrolyte resistance R₁, the polarization resistance or charge transfer resistance R_{ct}: (R₂−R₁), the constant phase element Q₁ (CPE) and Warburg element W₁ assigned to the diffusional resistance. The apparent electrode surface coverage parameter θ (%) is calculated as follows:

$$θ = \{ [ 1 - ( {R}_{ct 1} / {R}_{ct 2} ) ] \times 100 \} ,$$

wherein:
R_{ct1} is the value of the resistance to electron transfer through electrical double layer of the electrode after immobilization of the antibody;
R_{ct2} is the value of the resistance to electron transfer through electrical double layer after immobilization of the BSA.

The results obtained are shown in Tables 3, 4 and 5.

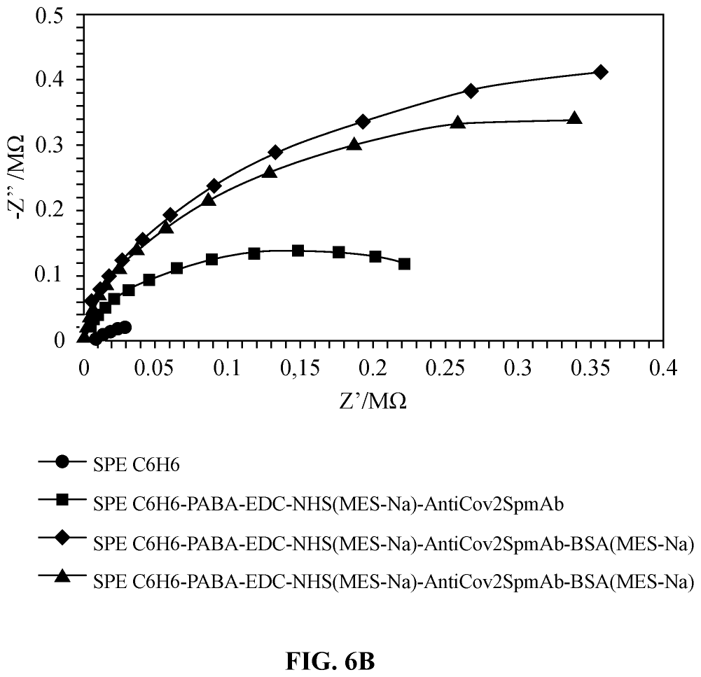

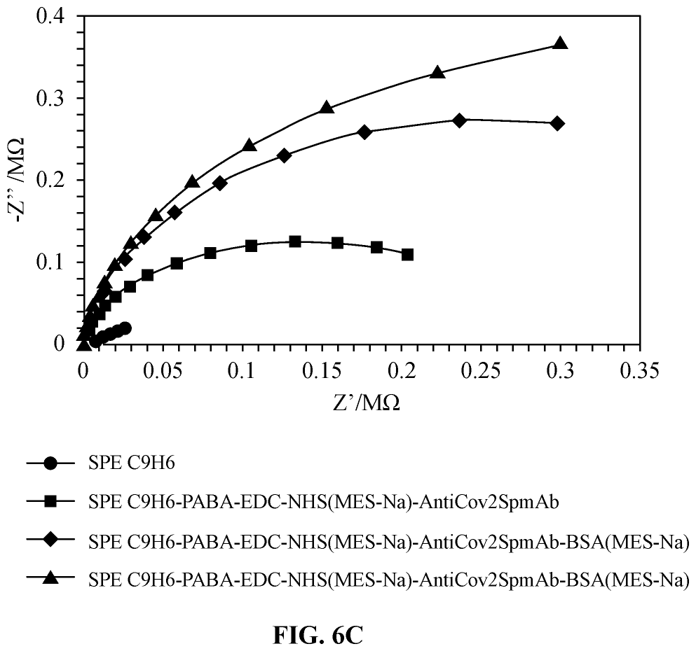

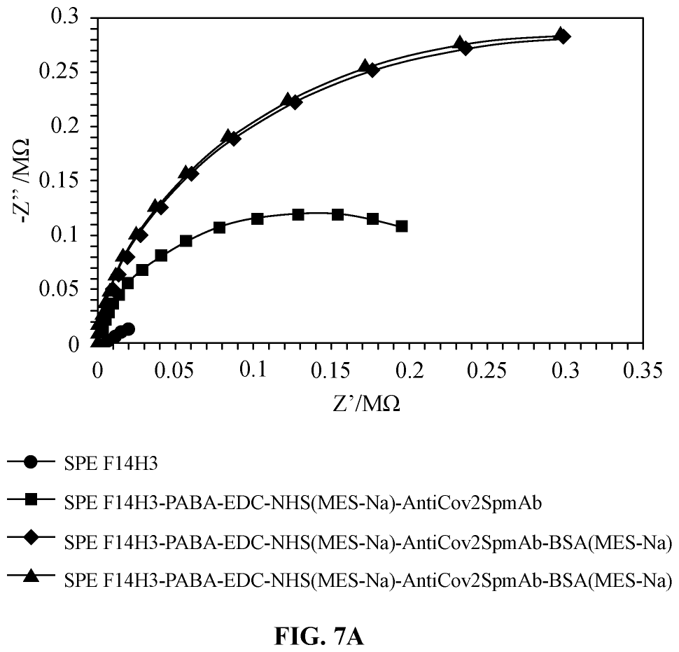

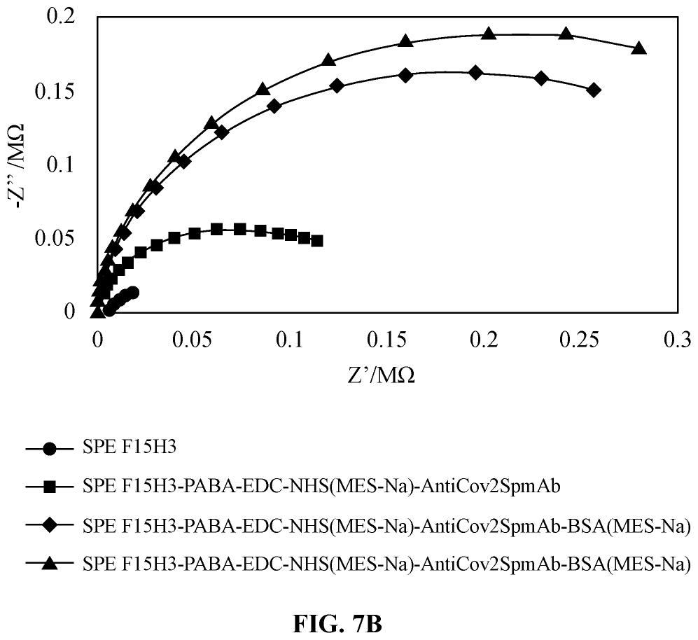

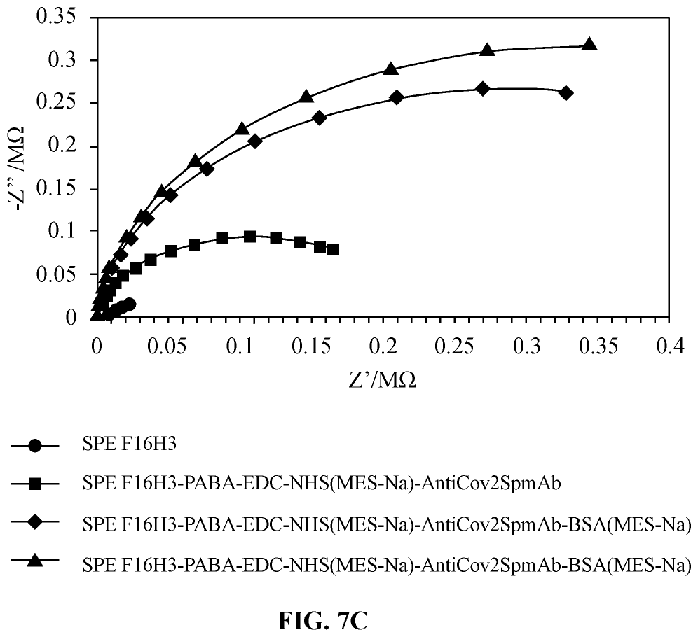

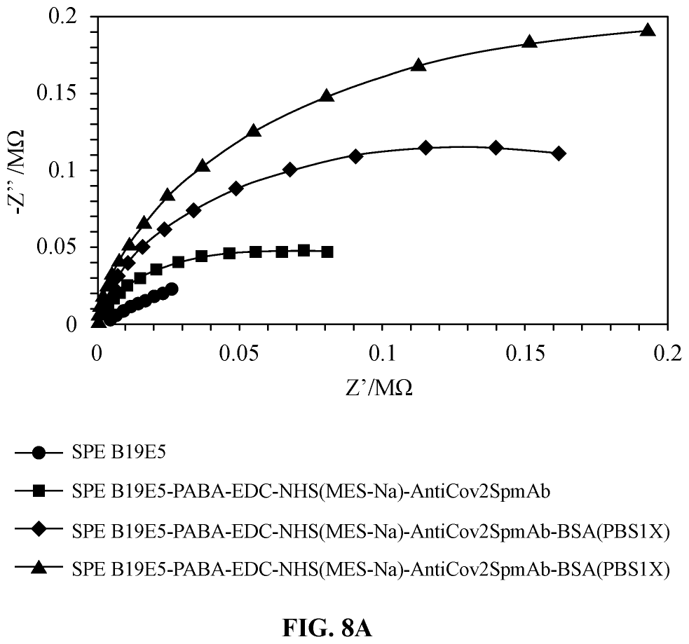

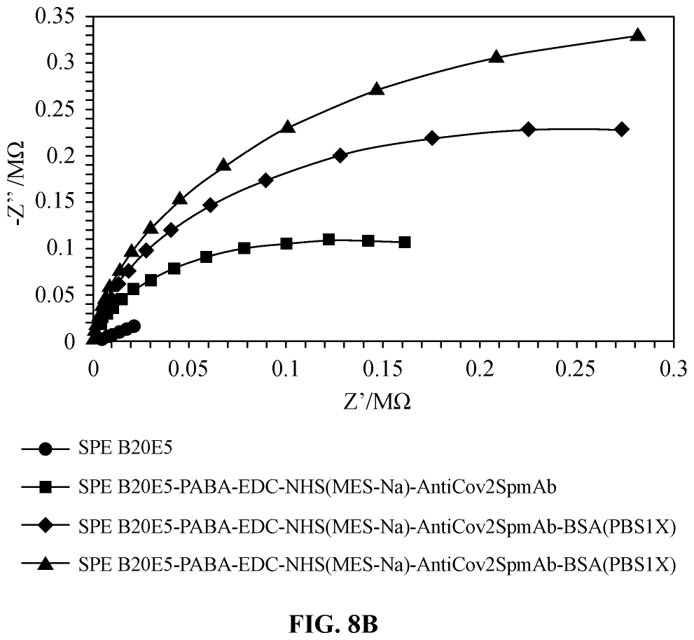

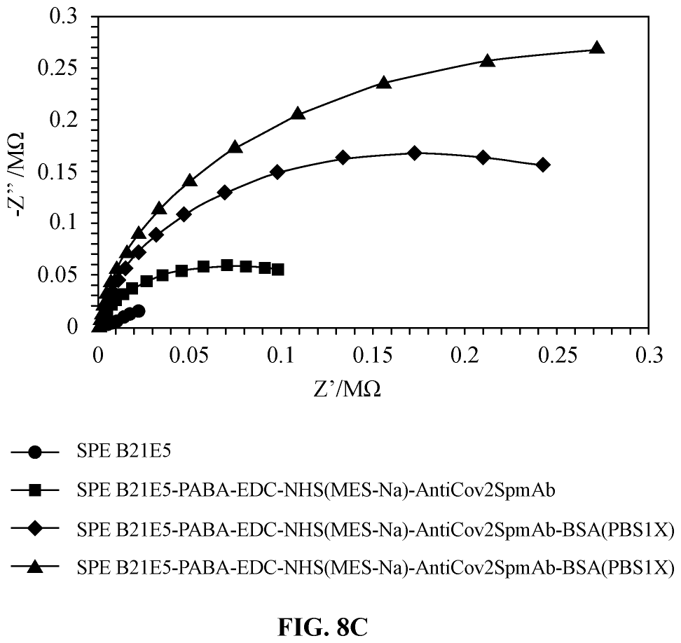

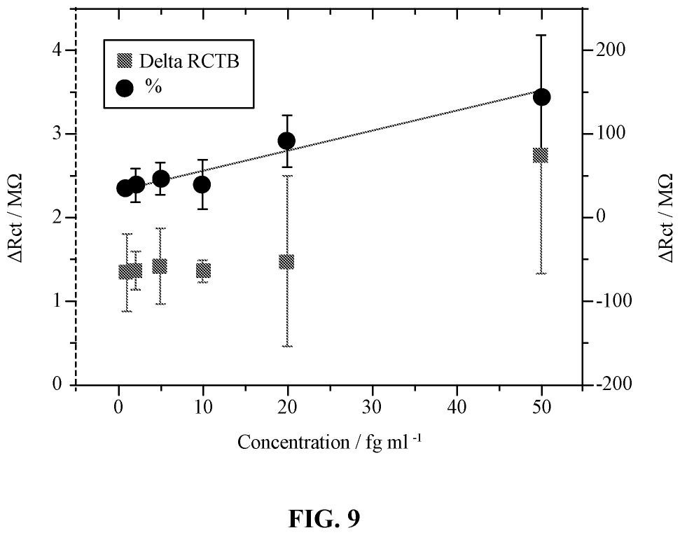

Table 4 and FIG. 6 show the results of C5H6, C6H6 and C9H6 electrodes, which were electrochemically modified with PABA at 2 cycles, and then BSA was immobilized in 0.5M MES pH 6.0 buffer. Activation with EDC/NHS (0.5M MES-Na pH 6.0) was performed at room temperature for 1 hour. Incubation of the antibody was performed at room temperature in a humid atmosphere for 2 hours. Incubation of BSA (0.5M MES-Na pH 6.0) was performed at room temperature for 1 hour. The incubation of the protein was performed at room temperature for 10 minutes.

Table 5 and FIG. 7 show the results of electrodes F14H3, F15H3, F16H3, which were electrochemically modified with PABA at 5 cycles, and subsequently immobilized BSA in 0.5M MES buffer pH 6.0. Activation with EDC/NHS (0.5M MES-Na pH 6.0) was performed at: 5.0° C.±1° C., for 1 hour. Incubation of the antibody was performed at: 5° C.±1° C., in humid atmosphere for 1 hour. Incubation of BSA (0.5M MES-Na pH 6.0) was performed at: 5.0° C.±1° C., for 1 hour. Protein incubation was performed at: 5.0° C.±1° C., for 10 minutes.

Table 6 and FIG. 8 show the results of electrodes B19E5, B20E5, B21E5, which were electrochemically modified with PABA at 2 cycles, and then BSA was immobilized in 1× PBS buffer (0.01M phosphate ion and pH 7.4). Activation with EDC/NHS (0.5M MES-Na pH 6.0) was performed at: 5.0° C.±1° C., for 1 hour. Incubation of the antibody was performed at: 5° C.±1° C., in humid atmosphere for 1 hour. Incubation of BSA (1×PBS) was performed at: 5.0° C.±1° C., for 1 hour. Protein incubation was performed at: 5.0° C.±1° C., for 10 minutes.

In Tables 4 and 5, it is evidenced that the difference in the value of the parameter |Δθ|, estimated on the first day and second day is between 0% and 6%. This variation suggests that the BSA (0.5M MES at pH 6.0) binds to the electrode surface by stable electrostatic interactions, specifically by hydrogen bonds or hydrogen bridges.

This can be related to the chemical environment or pH in which the BSA immobilization process takes place, its isoelectric point (IP) and the charge developed on the electrode surface: since the IP of BSA is 4.7 and it is dissolved in 0.5M MES buffer at pH 6.0, BSA will have a net negative charge (BSA has a net negative charge of −18 at pH 7.0). However, the electrode surface not covered with antibody, corresponding to the free or exposed carboxyl groups, would be protonated at pH 6.0, i.e., the —COOH form would predominate, favoring hydrogen bonding between the electrode surface and the amino acid residues on the BSA.

Additionally, favorable electrostatic interactions can develop between the negatively charged BSA and the bioconjugated human monoclonal antibody on the electrode surface, which has an isoelectric point between 6.6-7.2. Therefore, in 0.5M MES buffer at pH 6.0, the antibody will likely have a net positive charge.

According to the results obtained for the electrodes in Tables 4 and 5, at this stage, there would be no exposed or free carboxylic groups on the electrode surface, possibly because they were completely covered by the BSA during the immobilization process.

In contrast, the results obtained for the apparent coverage parameter of the electrode surface 0 (%), for the electrodes in Table 6, show that the value of difference of parameter |Δθ|, estimated for the first day and the second day is between 12% and 16%. This variation suggests that BSA (1×PBS at pH 7.4) does not bind efficiently to the electrode surface, since in 1×PBS solution at pH 7.4, the electrode surface corresponding to the exposed carboxyl groups would be deprotonated, i.e., the —COO— form would predominate and a predominantly negative charge would develop. Considering the isoelectric points mentioned before, both BSA and monoclonal antibody would have net negative charge at pH 7.4. Therefore, the increase of electrostatic repulsions is expected, disfavoring the effective immobilization of BSA on the electrode surface. This effect would possibly explain the variation of EIS values measured on different days on the electros in Table 6.

FIG. 9 shows the linear calibration curve derived from testing a set of biomodified electrodes as a function of the change in charge transfer resistance Rct (absolute and relative in percent) for 1 fg/ml, 2 fg/ml, 5 fg/ml, 10 fg/ml, 20 fg/ml and 50 fg/ml concentrations of SARS-COV-2 spike protein. These results further confirm a limit of detection (LOD) below 1 fg/ml. The statistical parameters are shown in Table 3.

TABLE 3
Statistical parameters of the calibration
curve of biomodified electrodes.

| | | |
|---|---|---|
| Parameter | Value | Standard Error |

| Residual sum of squares | | | 0.49952 | — |
|---|---|---|---|---|
| Pearson's r | | | 0.94862 | — |
| R-squared adjustment | | | 0.87484 | — |
| | D | Intercept | 32.97071 | 3.21393 |
| | | Regression slope | 2.37551 | 0.39619 |

TABLE 4
Measurements for electrodes C5H6, C6H6 and C9H6

| | | | | | | | Relative | Relative | Apparent | |
|---|---|---|---|---|---|---|---|---|---|---|
| | | | | | | | variation | variation | electrode | |
| | | | | | | | of electron | of electron | surface | |
| | | | | | | | transfer | transfer | coverage | |
| | | | | | | Rct/ | resistance. | resistance | (%)θ = | |
| | | | | | W1 | W(R2/ | ΔRct = | (%)ΔRct = | {[1 − (Rct1/ | Equivalent |
| | | Q1μ(Sⁿ/ | | | kΩ | W − | [(Rct2 − | {[[(Rct2 − Rct1)/ | Rct2)] × | electrical |
| Electrode | R1/W | Ωcm²) | n/Φ | R2/W | S⁻⁰^{_{—}}⁵ | R1/W) | Rct1)/Rct1]* | Rct1] × 100}*. | 100}* | circuit** |
| SPE C5H6 | 379.1 | 6.684 | 0.960 | 4.976 | 8.185 | 4,600 | N/A | N/A | N/A | R(Q[RW]) |
| SPE C5H6-PABA- | 329.9 | 11.09 | 0.966 | 120,000 | 14.34 | 120,000 | 25.03 | 2503% | N/A | R(Q[RW]) |
| EDC-NHS(MES- | | | | | | | | | | |
| Na)- | | | | | | | | | | |
| AntiCov2SpmAb | | | | | | | | | | |
| SPE C5H6-PABA- | 450.9 | 10.65 | 0.962 | 370,000 | 16.11 | 370,000 | 2.09 | 209% | 68% | R(Q[RW]) |
| EDC-NHS(MES- | | | | | | | | | | |
| Na) | | | | | | | | | | |
| AntiCov2SpmAb- | | | | | | | | | | |
| BSA(MES-Na) | | | | | | | | | | |
| SPE C5H6-PABA- | 362.9 | 11.27 | 0.960 | 380,000 | 21.99 | 380,000 | 0.03 | 3% | 68% | R(Q[RW]) |
| EDC-NHS(MES- | | | | | | | | | | |
| Na)- | | | | | | | | | | |
| AntiCov2SpmAb- | | | | | | | | | | |
| BSA(MES-Na) | | | | | | | | | | |
| SPE C6H6 | 402.7 | 8.926 | 0.91 | 6,298 | 8.088 | 590,000 | N/A | N/A | N/A | R(Q[RW]) |
| SPE C6H6-PABA- | 384.6 | 10.29 | 0.958 | 260,000 | 14.56 | 260,000 | 43.04 | 4304% | N/A | R(Q[RW]) |
| EDC-NHS(MES- | | | | | | | | | | |
| Na)- | | | | | | | | | | |
| AntiCov2SpmAb | | | | | | | | | | |
| SPE C6H6-PABA- | 381.1 | 9.586 | 0.967 | 790,000 | 29.38 | 790,000 | 2.04 | 204% | 67% | R(Q[RW]) |
| EDC-NHS(MES- | | | | | | | | | | |
| Na)- | | | | | | | | | | |
| AntiCov2SpmAb- | | | | | | | | | | |
| BSA(MES-Na) | | | | | | | | | | |
| SPE C6H6-PABA- | 410.7 | 10.03 | 0.965 | 670,000 | 21.36 | 670,000 | −0.15 | −15% | 61% | R(Q[RW]) |
| EDC-NHS(MES- | | | | | | | | | | |
| Na)- | | | | | | | | | | |
| AntiCov2SpmAb- | | | | | | | | | | |
| BSA(MES-Na) | | | | | | | | | | |
| SPE C9H6 | 423.2 | 7.646 | 0.966 | 4,491 | 7.417 | 4,070 | N/A | N/A | N/A | R(Q[RW]) |
| SPE C9H6-PABA- | 420.1 | 11.41 | 0.959 | 230,000 | 13.99 | 230,000 | 55.44 | 5544% | N/A | R(Q[RW]) |
| EDC-NHS(MES- | | | | | | | | | | |
| Na)- | | | | | | | | | | |
| AntiCov2SpmAb | | | | | | | | | | |
| SPE C9H6-PABA- | 477.7 | 11.09 | 0.961 | 560,000 | 16.85 | 560,000 | 1.44 | 144% | 59% | R(Q[RW]) |
| EDC-NHS(MES- | | | | | | | | | | |
| Na)- | | | | | | | | | | |
| AntiCov2SpmAb- | | | | | | | | | | |
| BSA(MES-Na) | | | | | | | | | | |
| SPE C9H6-PABA- | 398.0 | 11.20 | 0.972 | 650,000 | 38.43 | 650,000 | 0.16 | 16% | 65% | R(Q[RW]) |
| EDC-NHS(MES- | | | | | | | | | | |
| Na)- | | | | | | | | | | |
| AntiCov2SpmAb- | | | | | | | | | | |
| BSA(MES-Na) | | | | | | | | | | |

*Rct1 is the value of the resistance to electron transfer through the electric double layer of the bare electrode after antibody immobilization and BSA addition. Rct2 is the value of the resistance to electron transfer through the electric double layer, after antibody immobilization, BSA or protein addition.
**Equivalent electrical circuit R1 [Q1 (RctW)] containing electrolyte resistance R1, charge transfer resistance Rct (R2 − R1), constant phase element Q1 and Warburg element W1 assigned to the diffusional resistance. The constant phase element can be seen as a heuristic method to incorporate the surface heterogeneity effect along and across the electrode/electrolyte interface. Q has no physical meaning; it is just a parameter calculated when the circuit element behaves between the capacitor and the resistor.
The n (Φ) is not the phase shift here, but the degree to which the CPE is a resistor or capacitor. If n (Φ) is 0, CPE is just a resistor and if it is 1, CPE is a capacitor.

TABLE 5
Measurements for electrodes F14H3, F15H3 and F16H3

| | | | | | | | Relative | Relative | Apparent | |
|---|---|---|---|---|---|---|---|---|---|---|
| | | | | | | | variation | variation | electrode | |
| | | | | | | | of electron | of electron | surface | |
| | | | | | | | transfer | transfer | coverage | |
| | | | | | | Rct/ | resistance | resistance | (%)θ = | |
| | | | | | W1 | W(R2/ | ΔRct = | (%)ΔRct = | {[1 − (Rct1/ | Equivalent |
| | | Q1μ(Sⁿ/ | | | kΩ | W − | [(Rct2 − | {[(Rct2 − Rct1)/ | Rct2)] × | electrical |
| Electrode | R1/W | Ωcm²) | n/Φ | R2/W | S⁻⁰^{_{—}}⁵ | R1/W) | Rct1)/Rct1]* | Rct1] × 100}* | 100}* | circuit** |
| SPE F14H3 | 437.8 | 8.804 | 0.903 | 6,258 | 5.238 | 5,820 | N/A | N/A | N/A | R(Q[RW]) |
| SPE F14H3- | 397.7 | 11.88 | 0.952 | 230,000 | 12.55 | 230,000 | 38.45 | 3845% | N/A | R(Q[RW]) |
| PABA-EDC- | | | | | | | | | | |
| NHS(MES-Na)- | | | | | | | | | | |
| AntiCov2SpmAb | | | | | | | | | | |
| SPE F14H3- | 403.7 | 11.38 | 0.958 | 557,000 | 13.80 | 557,000 | 1.42 | 142% | 59% | R(Q[RW]) |
| PABA-EDC- | | | | | | | | | | |
| NHS(MES-Na)- | | | | | | | | | | |
| AntiCov2SpmAb- | | | | | | | | | | |
| BSA(MES-Na) | | | | | | | | | | |
| SPE F14H3- | 417.4 | 11.68 | 0.954 | 600,000 | 5.50 | 600,000 | 0.08 | 8% | 62% | R(Q[RW]) |
| PABA-EDC- | | | | | | | | | | |
| NHS(MES-Na)- | | | | | | | | | | |
| AntiCov2SpmAb- | | | | | | | | | | |
| BSA(MES-Na) | | | | | | | | | | |
| SPE F15H3 | 422.0 | 6.748 | 0.889 | 4,294 | 5.455 | 3,870 | N/A | N/A | N/A | R(Q[RW]) |
| SPE F15H3- | 391.5 | 10.82 | 0.943 | 100,000 | 10.90 | 99,600 | 24.73 | 2473% | N/A | R(Q[RW]) |
| PABA-EDC- | | | | | | | | | | |
| NHS(MES-Na)- | | | | | | | | | | |
| AntiCov2SpmAb | | | | | | | | | | |
| SPE F15H3- | 396.6 | 9.989 | 0.954 | 320,000 | 13.05 | 320,000 | 2.21 | 221% | 69% | R(Q[RW]) |
| PABA-EDC- | | | | | | | | | | |
| NHS(MES-Na)- | | | | | | | | | | |
| AntiCov2SpmAb- | | | | | | | | | | |
| BSA(MES-Na) | | | | | | | | | | |
| SPE F15H3- | 381.5 | 10.08 | 0.954 | 370,000 | 12.61 | 370,000 | 0.16 | 16% | 73% | R(Q[RW]) |
| PABA-EDC- | | | | | | | | | | |
| NHS(MES-Na)- | | | | | | | | | | |
| AntiCov2SpmAb- | | | | | | | | | | |
| BSA(MES-Na) | | | | | | | | | | |
| SPE F16H3 | 375.3 | 5.456 | 0.935 | 7,521 | 5.963 | 7,150 | N/A | N/A | N/A | R(Q[RW]) |
| SPE F16H3- | 351.7 | 10.42 | 0.953 | 170,000 | 13.48 | 170,000 | 22.74 | 2274% | N/A | R(Q[RW]) |
| PABA-EDC- | | | | | | | | | | |
| NHS(MES-Na)- | | | | | | | | | | |
| AntiCov2SpmAb | | | | | | | | | | |
| SPE F16H3- | 377.2 | 9.734 | 0.962 | 510,000 | 19.44 | 510,000 | 2.00 | 200% | 67% | R(Q[RW]) |
| PABA-EDC- | | | | | | | | | | |
| NHS(MES-Na)- | | | | | | | | | | |
| AntiCov2SpmAb- | | | | | | | | | | |
| BSA(MES-Na) | | | | | | | | | | |
| SPE F16H3- | 392.5 | 9.802 | 0.962 | 620,000 | 18.51 | 620,000 | 0.22 | 22% | 73% | R(Q[RW]) |
| PABA-EDC- | | | | | | | | | | |
| NHS(MES-Na)- | | | | | | | | | | |
| AntiCov2SpmAb- | | | | | | | | | | |
| BSA(MES-Na) | | | | | | | | | | |

*Rct1 is the value of the resistance to electron transfer through the electrical double layer of the bare electrode after antibody immobilization and BSA addition. Rct2 is the value of the resistance to electron transfer through the electric double layer after antibody immobilization, BSA or protein addition.
**Equivalent electrical circuit R1 [Q1 (RctW)] containing electrolyte resistance R1, charge transfer resistance Rct (R2 − R1), constant phase element Q1 and Warburg element W1 assigned to the diffusional resistance. The constant phase element can be seen as a heuristic method to incorporate the surface heterogeneity effect along and across the electrode/electrolyte interface. Q has no physical meaning; it is just a parameter calculated when the circuit element behaves between the capacitor and the resistor.
The n (Φ) is not the phase shift here, but the degree to which the CPE is a resistor or capacitor. If n (Φ) is 0, CPE is just a resistor and if it is 1, CPE is a capacitor.

TABLE 6
Measurements for electrodes B19E5, B20E5, B21E5

| | | | | | | | Relative | Relative | Electrode | |
|---|---|---|---|---|---|---|---|---|---|---|
| | | | | | | | variation | variation of | apparent | |
| | | | | | | | of electron | electron | surface | |
| | | | | | | | transfer | transfer | coverage | |
| | | | | | | Rct/ | resistance | resistance | (%)θ = | |
| | | | | | W1 | W(R2/ | ΔRct = | (%)ΔRct = | {[1 − (Rct1/ | Equivalent |
| | | Q1μ(Sⁿ/ | | | kΩ | W − | [(Rct2 − | {[(Rct2 − Rct1)/ | Rct2)] × | electrical |
| Electrode | R1/W | Ωcm²) | n/Φ | R2/W | S⁻⁰^{_{—}}⁵ | R1/W) | Rct1)/Rct1]* | Rct1] × 100}* | 100}* | circuit** |
| SPE B19E5 | 321.9 | 20.22 | 0.890 | 5,296 | 8.722 | 4,970 | N/A | N/A | N/A | R(Q[RW]) |
| SP B19E5-PABA-EDC- | 328.9 | 24.30 | 9.450 | 83,000 | 8.887 | 82,700 | 15.62 | 1562% | N/A | R(Q[RW]) |
| NHS(MES-Na)- | | | | | | | | | | |
| AntiCov2SpmAb | | | | | | | | | | |
| SP B19E5-PABA-EDC- | 373.1 | 16.94 | 0.965 | 200,000 | 14.23 | 200,000 | 1.41 | 141% | 59% | R(Q[RW]) |
| NHS(MES-Na)- | | | | | | | | | | |
| AntiCov2SpmAb- | | | | | | | | | | |
| BSA(PBS1X) | | | | | | | | | | |
| SP B19E5-PABA-EDC- | 349.0 | 17.06 | 0.973 | 330,000 | 21.77 | 330,000 | 0.65 | 65% | 75% | R(Q[RW]) |
| NHS(MES-Na)- | | | | | | | | | | |
| AntiCov2SpmAb- | | | | | | | | | | |
| BSA(PBS1X) | | | | | | | | | | |
| SPE B20E5 | 323.8 | 11.35 | 0.909 | 4,630 | 6.480 | 4,310 | N/A | N/A | N/A | R(Q[RW]) |
| SPE B20E5-PABA-EDC- | 325.8 | 15.24 | 0.947 | 200,000 | 13.59 | 200,000 | 45.37 | 4537% | N/A | R(Q[RW]) |
| NHS(MES-Na)- | | | | | | | | | | |
| AntiCov2SpmAb | | | | | | | | | | |
| SPE B20E5-PABA-EDC- | 340.6 | 11.83 | 0.959 | 460,000 | 12.09 | 460,000 | 1.30 | 130% | 57% | R(Q[RW]) |
| NHS(MES-Na)- | | | | | | | | | | |
| AntiCov2SpmAb- | | | | | | | | | | |
| BSA(PBS1X) | | | | | | | | | | |
| SPE B20E5-PABA-EDC- | 308.7 | 12.49 | 0.957 | 730,000 | 2.868 | 730,000 | 0.59 | 59% | 73% | R(Q[RW]) |
| NHS(MES-Na)- | | | | | | | | | | |
| AntiCov2SpmAb- | | | | | | | | | | |
| BSA(PBS1X) | | | | | | | | | | |
| SPE B21E5 | 306.6 | 12.29 | 0.912 | 6,284 | 6.440 | 5,980 | N/A | N/A | N/A | R(Q[RW]) |
| SPE B21E5-PABA-EDC- | 333.2 | 19.78 | 0.944 | 110,000 | 9.982 | 110,000 | 17.35 | 1735% | N/A | R(Q[RW]) |
| NHS(MES-Na)- | | | | | | | | | | |
| AntiCov2SpmAb | | | | | | | | | | |
| SPE B21E5-PABA-EDC- | 338.9 | 11.93 | 0.964 | 320,000 | 12.40 | 320,000 | 1.91 | 191% | 66% | R(Q[RW]) |
| NHS(MES-Na)- | | | | | | | | | | |
| AntiCov2SpmAb- | | | | | | | | | | |
| BSA(PBS1X) | | | | | | | | | | |
| SPE B21E5-PABA-EDC- | 275.5 | 12.58 | 0.967 | 500,000 | 20.30 | 500,000 | 0.56 | 56% | 78% | R(Q[RW]) |
| NHS(MES-Na)- | | | | | | | | | | |
| AntiCov2SpmAb- | | | | | | | | | | |
| BSA(PBS1X) | | | | | | | | | | |

*Rct1 is the value of the resistance to electron transfer through the electrical double layer of the bare electrode after antibody immobilization and BSA addition. Rct2 is the value of the resistance to electron transfer through the electric double layer after antibody immobilization, BSA or protein addition.
**Equivalent electrical circuit R1 [Q1 (RctW)] containing electrolyte resistance R1, charge transfer resistance Rct (R2 − R1), constant phase element Q1 and Warburg element W1 assigned to the diffusional resistance. The constant phase element can be seen as a heuristic method to incorporate the surface heterogeneity effect along and across the electrode/electrolyte interface. Q has no physical meaning; it is just a parameter calculated when the circuit element behaves between the capacitor and the resistor.
The n (Φ) is not the phase shift here, but the degree to which the CPE is a resistor or capacitor. If n (Φ) is 0, CPE is just a resistor and if it is 1, CPE is a capacitor.

1. A method for preparing a biomodified sensor for pathogen diagnostics, integrated in an electrochemical cell comprising a reference electrode, a counter electrode and one or more interdigitated planar working electrodes on a flexible substrate, comprising:
i) modifying the surface of a working electrode with a solution of an organic acid or a salt thereof;
ii) depositing on the modified surface a ligation solution comprising 1-ethyl-3-(3-dimethylaminopropyl) carbodiimide (EDC), a 2-morpholino ethane sulfonic acid buffer (MES) and N-hydroxysuccinimide (NHS) or N-hydroxysulfosuccinimide (sulfo-NHS);
iii) depositing on the surface containing the ligation solution, a solution comprising one or more types of antibodies;
iv) depositing on the surface containing the ligands with anchored antibodies, a concentrated solution of blocking agent dissolved in MES buffer solution;
wherein the one or more types of antibodies are selected from among antibodies that bind to one or more types of structural proteins of the pathogen, one or more types of structural proteins genetically conserved among different variants or lineages of the pathogen, or combinations thereof and,
wherein the electrode is selected between an electrode printed on a flexible polymer film, by screen printing with carbon allotrope material ink or a graphene electrode induced by CO₂ laser of a polyimide film.

2. The method of claim 1, wherein the organic acid or salt thereof is selected from pyrene butyric acid (PBA), 1-pyrenobutanoic acid succinimidyl ester (PBSE) and diazonium salt of p-aminobenzoic acid (PABA).

3. The method of claim 1, wherein the pathogen is a virus.

4. The method of claim 3 wherein the virus is severe acute respiratory syndrome-causing coronavirus type 2 (SARS-COV-2).

5. The method of claim 4, wherein the pathogen structural proteins, are selected from the group comprising: spike(S), nucleocapsid (N), membrane (M) or envelope (E) protein of SARS-COV-2 virus or mixtures thereof.

6. The method of claim 5, wherein the protein is S protein, N protein or a mixture thereof.

7. The method of claim 1, wherein each type of antibody is added at a concentration of 8 to 15 μg/ml.

8. The method of claim 1, wherein in step ii) the ligation solution is incubated at a constant temperature between 4 and 25° C. for 1 to 2 h.

9. The method of claim 1, wherein in step iii) the solution of one or more antibodies is incubated at a constant temperature between 4 and 25° C. for 1 to 2 h.

10. The method of claim 1, wherein in step iv) 4 μl of 1% (10 mg/ml) concentrated solution of bovine serum albumin (BSA) dissolved in a 0.5 M PBS or PBS/MES buffer solution at pH 6.0 is deposited and incubated at a constant temperature between 4 and 25° C. for 1 to 2 h.

11. A biomodified electrode for pathogen detection prepared by the method of claim 1.

12. The electrode of claim 10, wherein the antibody or antibodies selectively bind to one or more SARS-COV-2 structural proteins.

13. The electrode of claim 12, wherein the protein is selected from the group comprising SARS-COV-2 S protein and SARS-COV-2 N protein, or mixtures thereof.

14. Use of an electrode of any one of claims 10 to 12 for detecting pathogens in clinical or environmental samples.
